(19) United States
(12) Patent Application Publication
(10) Pub. No.: US 2006/0029563 A1
(43) Pub. Date: Feb. 9, 2006

(54) Cutanceous metabolic bio-activator

(75) Inventors: Jean-Noel Thorel, Paris (FR); Gerard Redziniak, Antony (FR)

Correspondence Address: OLIFF & BERRIDGE, PLC, P.O. BOX 19928, ALEXANDRIA, VA, 22320, US

(73) Assignee: Jean-Noel Thorel, Paris (FR)

(21) Appl. No.: 10/537,816

(22) PCT Filed: Dec. 23, 2003
(86) PCT No.: PCT/FR03/03883

§ 371 Date: Jun. 7, 2005

(30) Foreign Application Priority Data
Dec. 30, 2002 (FR) 02/16871
Dec. 30, 2002 (FR) 02/16872
Dec. 30, 2002 (FR) 02/16873
Dec. 30, 2002 (FR) 02/16874

(51) Int. Cl. A61K 8/64; A61K 38/08; A61K 38/05; A61K 38/04

(52) U.S. Cl. 424/70.14; 514/17; 514/18; 514/19

(57) ABSTRACT

The invention relates to a cosmetic composition comprising a bio-active system which combines (i) a stable form in aqueous solution of ATP (adenosine-tri-phosphate) with optionally an ATP precursor, e.g. Gp₄G (diguanosine tetraphosphate), or Ap₄A (diadenosine tetraphosphate), and (ii) at least one biomimetic peptide comprising at most six amino acids, mimicking a cutaneous polypeptide or a cutaneous protein, or a biomolecule which is agonist or antagonist in relation to the aforementioned polypeptide or protein. According to the invention, the composition takes the form of a water-in-oil or oil-in-water emulsion, the bio-active system being included in the aqueous phase.

The present invention relates in general to cosmetic compositions.

The object of the present invention is to implement, via the cosmetic route, a novel concept of cutaneous cell viability and/or stimulation, referred to by the term metabolic bioactivity. More particularly, the invention relates to a cutaneous metabolic bioactivator.

An individual's lifestyle, the aggressive environment, and degenerative chronobiological evolution result in the biological functions and vital faculties of skin tissues becoming weaker over time. It therefore appears to be essential to reestablish or maintain correct metabolic and catabolic functioning of skin cells (keratinocytes, Langerhans cells, melanocytes, fibroblasts, etc.) so that they exchange with their environment both exogenous energy and functional information.

An object of the present invention is therefore to increase or correct, naturally, the vast capacities of the skin by the combination of an exogenous supply of energy and of the stimulation of intercellular messages. The synergy between this supply and this stimulation allows the skin to react against any attack or any disfunction, by activating preexisting metabolic mechanisms in the skin (molecular, cellular, tissue mechanisms) and optimizing, where appropriate, the interaction between the skin and the cosmetic active agent(s) provided by dermo-cutaneous care or treatments.

To this end, the present invention proposes a cosmetic composition comprising a bioactive system that combines, firstly, a stable form, in aqueous solution, of ATP (adenosine triphosphate) with, optionally, an ATP precursor, for example Gp₄G (diguanosine tetraphosphate) or AP₄A (diadenosine tetraphosphate) and, secondly, at least one biomimetic peptide comprising at most six amino acids, that mimics a cutaneous polypeptide or a cutaneous protein, or a biomolecule that is an agonist or antagonist with respect to said peptide or to said protein.

The term “cosmetic composition” is intended to mean any composition whose function is to maintain, restore or improve the appearance of the superficial parts of the human body, mainly of the skin, whatever the method of administration of said composition, i.e. via external topical administration or via internal oral administration.

The term “ATP precursor” is intended to mean any biochemical compound that is an intermediate in the de novo biosynthesis of ATP; preferably, the ATP precursor is Gp₄G (or diguanosine tetraphosphate) or Ap₄A (diadenosine tetraphosphate).

The term “biomimetic peptide” is intended to mean any peptide comprising at most six amino acids, that mimics a cutaneous peptide or a cutaneous protein, or a biomolecule that is agonist or antagonist with respect to said peptide or to said protein, which peptide or proteins plays a role in or is involved in a cutaneous biosynthesis or the transfer of cutaneous information.

Preferably, mimic peptides that are selected include the peptides or proteins that modulate the properties of the skin and immunity.

By way of example of the peptides or proteins of the skin that are mimicked by the peptides belonging to the bioactive system according to the present invention, the following are selected:
1) neuromediators, including catecholamines (dopamine, adrenaline, noradrenaline), endorphins (for example beta-endorphin) and enkephalins (for example met-enkephalins); by way of example, the following are selected:
   somatostatin; cf. SEQ ID No. 3,
   β-CGRP peptide; cf. SEQ ID No. 6,
   β-endorphin; cf. SEQ ID No. 9,
   leu-enkephalin; cf. SEQ ID No. 10,
   met-enkephalin; cf. SEQ ID No. 11.
2) neuropeptides, for example:
   substance P; cf. SEQ ID No. 1,
   neuropeptide Y; cf. SEQ ID No. 2,
   neurotensin; cf. SEQ ID No. 4,
   α-CGRP peptide (calcitonin gene related peptide); cf. SEQ ID No. 5,
   neurokinins A and B,
   GRP peptide (gastrin releasing peptide); cf. SEQ No. 7,
   bradykinin; cf. SEQ ID No. 8.
3) neurohormones, for example:
   α-MSH (melanocyte stimulating hormone) peptide; cf. SEQ ID No. 12,
   ACTH (adrenocorticotrophic hormone) peptide; cf. SEQ ID No. 13,
   “prolactin releasing” peptide; cf. SEQ ID No. 14.

By way of example, a biomimetic peptide used according to the present invention mimics a peptide that is a substance P antagonist or a CGRP peptide antagonist.

By way of example, a biomimetic peptide used according to the present invention mimics a peptide that is a somatostatin agonist.

By way of example, a biomimetic peptide used according to the present invention mimics a peptide that is a neuropeptide Y antagonist or modulator.

By way of example, a biomimetic peptide used according to the present invention mimics a peptide that is a bradykinin receptor antagonist.

By way of example, a biomimetic peptide used according to the present invention mimics a peptide that is an α-MSH agonist or antagonist.

The term “mimic” or “mimicry” is intended to mean the characteristic according to which the peptide under consideration exerts, in vitro, and in particular in a composition according to the invention, a biological effect similar or close to a biological function in vivo (for example in the skin) of a reference biomolecule (for example peptide or protein).

Throughout the description and the claims, the term “peptide” should be understood to mean both a series of several unsubstituted amino acids and a series of the same amino acids in which some, for example the N- terminal amino acid and/or the C-terminal amino acid, are substituted with a group or substituent, that may or may not be functional.

These peptides can be obtained either by conventional chemical synthesis (in solid phase or in homogeneous liquid phase), or by enzymatic synthesis (Kullman et al., J. Biol. Chem. 1980, 255, 8234) from the constitutive amino acids or from derivatives thereof.

These peptides can also be obtained by fermentation of a bacterial strain that may or may not be modified by genetic engineering, so as to produce the desired sequences or their various fragments.

Finally, these peptides can be obtained by extraction of proteins of animal or plant, preferably plant, origin, followed by a controlled hydrolysis that releases the peptides in question. Many proteins found in plants are liable to contain advantageous sequences within their structure. Controlled hydrolysis makes it possible to free these peptide fragments.

In accordance with the present invention and according to a first variant, the bioactive system selected in the cosmetic composition constitutes, by itself, the active principle of the latter.

In this case, the biomimetic peptide selected makes it possible to “functionalize” the cosmetic composition; for example:
by selecting mimicry with α-MSH, a pigmenting activity is obtained; conversely, by selecting mimicry with an α-MSH antagonist, depigmenting activity is obtained;
by selecting mimicry with a peptide that is a substance P antagonist, a soothing effect is obtained;
by selecting mimicry with a peptide that is a CGRP peptide antagonist, an effect that inhibits irritations of neurogenic origin is obtained;
by selecting mimicry with a peptide that is a bradykinin antagonist, an effect that inhibits any intolerance or sensitization is obtained.

According to a second variant, the bioactive system selected in the cosmetic composition potentiates one or more active principles, that are present in said composition.

By virtue of the invention, the bioactive system that characterizes the latter makes it possible both to restore and/or to maintain the natural activity of the epidermis. This is particularly borne out if the cosmetic composition also contains skin nutrients and/or an aqueous phase that ensures viability of the skin cells.

The present invention also provides the following embodiments:
the stable form of ATP is a sodium salt, for example a disodium salt of ATP,
the biomimetic peptide is functionally active in the biosynthesis of a structural molecule of the skin or of an enzyme present in the skin,
the biomimetic peptide is functionally active in the transfer of information in the skin, and is, for example, a biologically active fraction of a hormone or a cytokine present in the skin,
the biomimetic peptide is chosen from the group consisting of histidine-β-alanyl (mimics superoxide dismutase), the peptide R-Gly-Gln-Pro-Arg, the peptide Tyr-Arg, the peptide R-Lys-Thr-Thr-Lys-Ser, and N-acetyl-Tyr-Arg-R (mimics endorphins); R being any amino acid; the peptide Lys-Thr-Thr-Lys-Ser, the peptide Ala-Arg-His-Leu-Phe-Tyr (mimics alpha-MSH), and the peptide Gly-Gln-Asp-Pro-Val-Lys (mimics elafin),
the biomimetic peptide is, for example, a dipeptide; in this regard, the dipeptide may correspond to the formula Arg-R or His-R, in which R is any amino acid; or else the dipeptide may be in the form of an oligomer, of formula (R—R)ₙ, with 1<n<3; for example, the dipeptide corresponds to the formula (Arg-Lys)ₙ, with 1<n<3,
the biomimetic peptide is a tripeptide, for example corresponding to the formula Gly-His-Lys, Gly-Glu-Pro or Lys-Pro-Val,
the biomimetic peptide is a tetrapeptide, for example corresponding to the formula Leu-Pro-Thr-Val, Lys-Thr-Ser-R or Gly-Glu-Pro-R; R being any amino acid,
the biomimetic peptide is a pentapeptide, for example Val-Ala-Lys-Leu-R; R being any amino acid,
the biomimetic peptide is a hexapeptide; by way of example, the biomimetic peptide is Ala-R₁-R₂-R₃-Phe-Try, with R₁, R₂ and R₃ each being equal to any amino acid,
the cosmetic composition may comprise an amino acid, alongside the bioactive system, said amino acid being chosen, for example, from the group consisting of creatine, decarboxy carnosine, and a glutamine, for example N-acetylglutamine,
the cosmetic composition according to the invention may comprise, alongside the bioactive system, a protein, for example chosen from the group consisting of superoxide dismutase, endonucleases, photolyase, and cytokines from milk,
the bioactive system represents at most 10%, and preferably between 1% and 10⁻⁷%, by weight of said composition,
the cosmetic composition according to the invention comprises at least one cosmetic active principle potentiated by the bioactive system according to the invention,
conventionally, the cosmetic composition is in the form of a water-in-oil or oil-in-water emulsion, the bioactive system being included, for example in solution, in the aqueous phase,
in the bioactive system according to the invention, the ATP and, optionally, the ATP precursor represent at most 10%, and preferably between 0.01% and 5%, by weight.

The effective amount of active agent(s) optionally present in a composition according to the invention corresponds to the amount required to obtain the desired result, and the compositions according to the invention depend on the use for which these compositions are intended.

A first category comprises the cosmetic compositions intended to be applied to healthy skin in order to improve the esthetics and the comfort thereof. Healthy skin can be defined as free of pathology, but without however being in perfect condition. This skin may have signs of dryness, signs of irritation, wrinkles related to chronological or actinic aging, regions of hypersecretion of sebum, or regions of hypopigmentation or of hyperpigmentation. In addition, healthy skin may need temporary or permanent photoprotection in order to withstand sunlight better.

Another category comprises the compositions intended to be applied to skin that has been made vulnerable by disease or medications, either in a preventive capacity or as a treatment continuing on from medical treatments.

The cosmetic compositions according to the present invention may also comprise at least one cosmetic active agent chosen from antioxidants, free-radical scavengers, α-hydroxy acids, vitamins, sunscreens or filters, insect repellents and anti-inflammatories.

Of course, those skilled in the art will take care to choose this or these optional active agent (s), and/or its (their) amount(s), in such a way that the advantageous properties of the bioactive system according to the invention are not, or are not substantially, altered by the envisioned addition(s). Preferably, a synergy between the bioactive system according to the invention and the active agent(s) under consideration will be sought.

The compositions of the invention may be prepared according to techniques well known to those skilled in the art, in particular those intended for preparing emulsions of oil-in-water (O/W) or water-in-oil (W/O) type.

These compositions may in particular be provided in the form of a simple emulsion or a complex emulsion: double (O/W or W/O) or triple (W/O/W or O/W/O), such as a cream, a milk, a gel or a cream-gel; of a powder or of a solid tube, and may optionally be packaged as an aerosol and be provided in the form of a mousse or of a spray.

When the cosmetic composition according to the invention is used for the protection or the care of human epidermis, or as an antisun composition, it may be provided in the form of a suspension or of a dispersion in solvents or fatty substances, or in the form of a nonionic vesicular dispersion, or else in the form of an emulsion, preferably of oil-in-water type, such as a cream or a milk, in the form of an ointment, of a gel, of a cream-gel, of a solid tube, of a powder, of a stick, of an aerosol mousse or of a spray.

When the cosmetic composition according to the invention is used for protecting the hair, it may be in the form of a shampoo, of a lotion, of a gel, of an emulsion, or of a nonionic vesicular dispersion, and may constitute, for example, a rinse-out composition, to be applied before or after shampooing, before or after dyeing or bleaching, before, during or after permanent-waving or straightening the hair; this composition may also be provided in the form of a styling or treating lotion or gel, a blow-drying or hairsetting lotion or gel or a composition for permanent-waving, straightening, dyeing or bleaching the hair.

When the composition is used as a makeup product for the nails, the eyelashes, the eyebrows or the skin, such as an epidermal treatment cream, a foundation, a tube of lipstick, an eyeshadow, a blusher, a mascara or an eyeliner, it may be provided in solid or pasty, anhydrous or aqueous form, such as oil-in-water or water-in-oil emulsions, nonionic vesicular dispersions or alternatively suspensions.

For the compositions according to the invention, the pH will be physiological, between 4 and 7. When it is applied topically, the composition comprising at least one ATP and, optionally, a precursor, conjugated to at least one biomimetic peptide, can be applied to the face, the neck, the scalp, the mucous membranes, the nails, the body, the chest, the feet, the legs, or any other part of the body.

The compositions of the invention may also comprise conventional cosmetic adjuvants, in particular chosen from fatty substances, organic solvents other than those used specifically in the context of the present invention, emulsifiers, ionic or nonionic thickeners, soothing agents, opacifiers, stabilizers, emollients, silicones, antifoaming agents, moisturizers, vitamins, fragrances, preserving agents, surfactants, fillers, polymers, propellants, alkalifying or acidifying agents, dyes or any other ingredients normally used in cosmetics, in particular for producing compositions in the form of emulsions. By way of example, the compositions according to the invention comprise screening or reflecting agents, in the case of products intended to be used in the outside environment and in the sun.

The fatty substances may consist of an oil or a wax, or mixtures thereof, and they also comprise fatty acids, fatty alcohols and fatty acid esters. The oils may be chosen from animal, plant, mineral or synthetic oils, and in particular from liquid petroleum jelly, paraffin oil, volatile or nonvolatile silicone oils, isoparaffins, polyolefins, and fluoro and perfluoro oils. Similarly, the waxes may be chosen from animal, fossil, plant, mineral or synthetic waxes that are known in themselves.

Among polar oils, mention may be made of the oil known as “Finsolv TN”, tridecyl trimellitate, isononyl isononanoate, isopropyl myristate, decaprylyl carbonate, or Guerbet alcohol benzoates and hydroxy benzoates, such as the product called “Hallbrite BHB” from the company CP Hall Company.

By way of indication, for the antisun formulations in accordance with the invention that have an oil-in-water emulsion-type carrier, the aqueous phase (comprising in particular the hydrophilic screening agents) generally represents from 50 to 95% by weight, preferably from 70 to 90% by weight, relative to the entire formulation, the oily phase (comprising in particular the liophilic screening agents) represents from 5 to 50% by weight, preferably from 10 to 30% by weight, relative to the entire formulation, and the (co)emulsifier(s) represent(s) from 0.5 to 20% by weight, preferably from 2 to 10% by weight, relative to the entire formulation.

In particular, the compositions according to the present invention can be obtained in -the form of -an anhydrous composition which has transparency and translucency properties that are entirely noteworthy.

A subject of the present invention is also the use of a cosmetic composition according to the present invention, for producing a skin care product, a makeup product for the skin, the lips and/or the integuments, and an antisun product, and of a dermatological composition for skin care and/or treatment.

The composition according to the invention may be in the form of a tinted dermatological or care composition for keratin materials such as the skin, the lips and/or the integuments, in the form of an antisun composition or a body hygiene composition, in particular in the form of a deodorant product or a makeup-removing product, in stick form. It may, in particular, be used as a care base for the skin, the integuments or the lips (lip balms, for protecting the lips against the cold and/or the sun and/or the wind, or a care cream for the skin, the nails or the hair).

The composition of the invention may also be in the form of a colored makeup product for the skin, in particular a foundation, optionally having care or treatment properties, a blusher, a makeup rouge, an eyeshadow, a concealer product, an eyeliner, a body makeup product; a lip makeup product such as a lipstick, optionally having care or treatment properties; a makeup product for the integuments such as the nails or the eyelashes, in particular in the form of a mascara cake, or for the eyebrows and the hair, especially in the form of a pencil. In particular, the composition of the invention may be a cosmetic product containing, besides the bioactive system, cosmetic and/or dermatological active agents.

A cosmetic composition according to the invention may also comprise pearlescent agents, pigments, or alternatively nanopigments (mean size of the primary particles: generally between 5 nm and 100 nm, preferably between 10 nm and 50 nm) of metal oxides that may or may not be coated, for instance nanopigments of titanium oxide (amorphous or crystallized in rutile and/or anatase form), of iron oxide, of zinc oxide, of zirconium oxide or of cerium oxide, and mixtures thereof, which are all UV-photoprotective agents well known in themselves. Conventional coating agents are, moreover, alumina and/or aluminum stearate. Such coated or uncoated metal oxide nanopigments are in particular described in patent applications EP-A-0518772 and EP-A-0518773.

Of course, the composition of the invention should be cosmetically or dermatologically acceptable, i.e. it should contain a nontoxic physiologically acceptable medium and should be able to be applied to the skin, the integuments or the lips of human beings. For the purpose of the invention, the term “cosmetically acceptable” is intended to mean a composition with a pleasant appearance, odor and feel.

### Trials

Trial No. 1

The biosimulatory activity of the L-citrullyl-Larginine peptide, associated or not associated with ATP, in the form of a disodium salt, on the cellular metabolism of normal human keratinocytes is evaluated.

1. Principle

The procedure consists in measuring the amounts of ATP in a culture of fibroblasts in serum-deprived (2%) medium, in comparison with:
a medium enriched with citrullyl-arginine, at variable concentrations,
a medium enriched with ATP, at variable concentrations,
a medium enriched with citrullyl-arginine and ATP, at variable concentrations.

The aim of this trial is to evaluate the activating effect of the mixture studied, associated or not associated with ATP disodium salt, with respect to ATP synthesis by normal human keratinocytes.

2. Method

Cell Culture

The trial is carried out on an in vitro culture of normal human keratinocytes seeded at a density of 10⁵ cells/well in 6-well plates.

L-citrullyl-L-arginine

In order to determine the concentrations applicable in the context of the trial, a prior cell viability test is carried out on normal human keratinocytes.

L-citrullyl-L-arginine is dissolved in water and the final concentration is fixed at 0.1%.

6 dilutions of L-citrullyl-L-arginine from 10⁻⁴% to 10⁻²% are brought into contact with the cells for 24 h and 48 h. A “water” condition is realized as a control.

For the trial, the L-citrullyl-L-arginine will be tested at the highest 2 concentrations that allow the cell viability to be maintained at a level greater than 80%, after 48 h of contact, i.e. 0.0001% and 0.01%.

UV B Irradiation

The keratinocytes, seeded in 6-well plates at a density of 10⁵ cells/well, are cultured in standard medium (Epilife Sigma) for 48 h. Before irradiation, the culture medium is removed, the cells are rinsed with PBS buffer, and 1 ml thereof is left in contact with the cells for the irradiation. The keratinocytes are subjected to a UV B irradiation (312 nm) of 20 mJ/cm². An identical nonirradiated condition is realized in parallel.

After irradiation (and on the nonirradiated parallel), the PBS is removed and the cells are placed under the various conditions studied:
control of water in the culture medium,
0.001% L-citrullyl-L-arginine in the culture medium,
0.01% L-citrullyl-L-arginine in the culture medium.

Each condition is realized in triplicate.

After contact for 24 h or 48 h after irradiation, the conditioned culture supernatants are recovered. The amount of endothelin-1 secreted by the keratinocytes is measured on 100 μl of supernatant by means of an ELISA technique (R&D Systems, Abingdon, UK). The endothelin-1 levels are calculated by means of the standard curve prepared with synthesized human endothelin-1. The results are expressed in pg of endothelin-1 standardized with respect to the protein concentration (pg ET-1/mg of proteins).

3. Results

The application of L-citrullyl-L-arginine for 24 h at the lowest concentration tested (0.0001%) results in an increase in the basal secretion of endothelin-1, by the keratinocytes (nonirradiated condition), of 20% compared with the “water” control. At the higher concentration (0.01%), this activating effect is much more marked: 70%.

The UV B irradiation results in a 50% stimulation of the secretion of endothelin-1 by the keratinocytes under the “water” control condition. After contact 24 h with 0.0001% L-citrullyl-L-arginine, the secretion of endothelin-1 is increased by 6% compared with the irradiated “water” control. At 0.001%, the activating effect of the L-citrullyl-L-arginine is greatly increased: 28%.

The application of L-citrullyl-L-arginine for 48 h at the lowest concentration tested (0.0001%) results in an increase in the basal secretion of endothelin-1, by the keratinocytes (nonirradiated condition), of 26% compared with the “water” control. At the higher concentration: 0.01%, this activating effect is much more marked: 82%.

The UV B irradiation results in a 40% stimulation of the secretion of endothelin-1 by the keratinocytes under the “water” control condition. After contact for 48 h with 0.0001% L-citrullyl-L-arginine, the secretion of endothelin-1 is increased by 4% compared with the irradiated “water” control. At 0.01%, the activating effect of the L-citrullyl-L-arginine is greatly increased: 63%.

### 4. CONCLUSION

Under the experimental conditions thus defined, for the dilutions and incubation times chosen, it is found that:

L-citrullyl-L-arginine, at a concentration of 0.01%, exerts a considerable activating effect on the secretion of endothelin-1 by normal human keratinocytes.

In the presence of 0.01% ATP, the activating effect is stimulated by 20%. There is clearly a synergistic effect between the molecules of ATP and of the L-citrullyl-L-arginine peptide.

The same studies carried out with ATP alone do not show any effect on endothelin-1 synthesis.

Trial No. 2

The effect of the combination ATP+dipeptides on the growth of normal human fibroblasts is studied.

The products studied are:
ATP, in the form of the disodium salt,
β-alanyl-L-histidine (carnosine),
citrullyl-arginine (exsy algine).
   1. Objective of the Study

The objective of this trial is to evaluate the effect of the combination ATP+peptides, added to a culture medium, on the growth of an immortalized line of human fibroblasts, HaCaT cells.

In this study, two peptides are simultaneously and/or concomitantly combined with ATP: β-alanyl-L-histidine (Dragoco) and L-citrullyl-L-arginine (Exsymol)

The study is carried out on a culture prepared in the standard culture medium for HaCaT cells, DMEM (Sigma) in the presence of fetal calf serum (SVF) at 2 or 10%.

2. Techniques

The HaCaT cells are seeded at low density in a 96-well plate in the standard medium (DMEM+10% SVF) and grow for 24 h after seeding in this medium.

On the 2nd day, the cells are placed under the various conditions studied.

The test concentrations in terms of ATP, β-alanyl-L- histidine (Carnosine) and L-citrullyl-L-arginine (exsy- algine) are determined subsequent to preliminary cytotoxicity studies.

The following conditions are prepared:
A control condition: culture medium alone+SVF
A condition consisting of β-alanyl-L-histidine alone at 0.5%.
A condition consisting of L-citrullyl-L-arginine are known at 0.5%.
A condition consisting of ATP alone at 1 μg/ml.
A condition consisting of ATP (1 μg/ml)+β-alanyl-L-histidine (0.5%).
A condition consisting of ATP (1 μg/ml)+L-citrullyl-L-arginine (0.5%)
A condition consisting of ATP (1 μg/ml)+β-alanyl-L-histidine (0.5%)+L- citrullyl-L-arginine (0.5%).

These various conditions are prepared both in the DMEM containing 2% SVF and the DMEM containing 10% SVF.

Each condition is prepared in triplicate. The media are not renewed in the course of the experiment.

The cell density is evaluated 24 h after seeding of the cells, before the cells are brought into contact with the various study conditions (=T0), and the growth of the HaCaT cells is then evaluated on the 2nd, 5th and 7th days of culture by means of the WST-1 conversion method (reading at 450 nm).

3. Results

The cell growth is objectified by measuring the cell viability at various times of the experiment. The results obtained give the percentage cell viability calculated with respect to the initial cell density at T0, for which it is estimated that the cell density is equal to 100%. The effects of the various products on cell growth are analyzed at the various times of the experiment, relative to the nontreated control at the same time of the experiment.

After 2 days of culture, a lack of growth with maintenance of cell viability is observed for the control condition, relative to the TO control, which is explained by the switch into growth factor depleted medium (2% SVF).

The addition of β-alanyl-L-histidine alone at a concentration of 0.5% greatly stimulates the cell growth (+22% relative to the nontreated control). At a concentration of 0.1%, no effect on growth is observed.

The addition of ATP alone substantially decreases cell viability.

The addition of ATP to the β-alanyl-L-histidine results in a suppression of the inhibitory effect of the ATP on the cell viability and stimulates the latter beyond the level obtained with β-alanyl-L-histidine alone, for the 2 concentrations tested.

The addition of L-citrullyl-L-arginine alone, at a concentration of 0.1% and 0.5%, does not stimulate cell growth (+5%) relative to the nontreated control.

After 5 days of culture, a slight decrease in cell viability is observed, relative to T0, due to the culture being maintained in growth factor-depleted medium.

The stimulatory effect on cell growth of β-alanyl-L-histidine at a concentration of 0.5% (+12%) is again observed.

The addition of ATP alone substantially decreases cell viability, which effect disappears subsequent to the addition of 0.5% β-alanyl-L-histidine.

L-citrullyl-L-arginine alone still does not stimulate cell growth, whatever the concentration used.

### 4. CONCLUSION

Under the experimental conditions thus defined and at the concentrations tested, it appears that the addition of ATP alone substantially decreases the viability of normal human fibroblasts after 2 to 5 days of contact (−5 to −10%).

The combination ATP+β-alanyl-L-histidine cancels the inhibitory effect of the ATP on cell viability and stimulates the latter beyond the level obtained with β-alanyl-L-histidine alone, which exerts a stimulatory effect on cell growth at a concentration of 0.5%.

The combination ATP+L-citrullyl-L-arginine exerts no stimulatory effect on cell growth, whatever the concentration used.

Trial No. 3

1. Cell Culture

Cultures of normal human melanocytes (MHNs) are prepared from fore skins of infants and of newborns suffering from phimosis. The melanocytes obtained from skin fragments are placed in MCDB 153 medium (Sigma St Louis, Mo., USA) supplemented with 30 μg/ml of bovine pituitary extract (BPE) (Life Technologies, Paisley, England), 2% of fetal calf serum (SVF) (Dominique Dutscher, Brumath, France), 16 nM of phorbol-12-myristate-13-acetate (Sigma), 5 μg/ml of insulin and 1.1 μM of hydrocortisone (Sigma). The cultures are maintained in an incubator at 37° C. and-in-an atmosphere containing 5% CO₂. Pure cultures of melanocytes are obtained after 2 to 3 weeks.

2. Contact Time, Irradiation of Cells and Preparation of Slides

All the compositions tested are solubilized in DMSO at the maximum concentrations permitted. Preliminary trials are carried out in order to determine the subtoxic concentrations on the keratinocytes. The final concentration of DMSO is always less than 2%.

The melanocytes are brought into contact with the product for 30 min at 37° C., and are then irradiated with UVA radiation. The UVA irradiations are generated by means of a Bio-Sun UV-irradiator (Vilbert Lourmat, Marne la Vallée). This device is equipped with monochromatic lamps that emit a wavelength of 312 nm and/or 365 nm. The lamps deliver a calculated energy by means of an RMW-365/312 radiometer. The energies delivered are 0.8 J/cm² for the UVA range. The “comet” assay is carried out immediately after exposure. Two types of controls are included in these experiments:
negative controls: melanocytes that are not treated but are irradiated with UVA radiation; melanocytes treated for 30 min with the composition tested, but not irradiated.
Positive control: melanocytes that are not treated but are irradiated.

After treatment with a typsin/EDTA mixture (0.05%/0.02%) for 2 to 3 minutes, the cultures are 15 recovered by centrifugation and placed in PBS buffer without Ca++ and without Mg++ (Sigma). Following a second centrifugation, the cells (4.5-5.0×10⁴ cells) are suspended in 0.5% low melting point (LMP) agarose (Sigma). The mixture is directly deposited onto microscope slides coated with a prelayer of agarose (1.6%) dried overnight at ambient temperature and freshly precoated with a second layer of agarose (0.8%).

3. The Comet Assay (Dry Slide Technique) and Enzymatic Treatment

The protocol for the comet assay is that of De Meo et al. [1], incorporating the “dry slide” technique [2]. After the irradiations, the slides are placed in a lysis bath (2.5 M NaCl, 100 mM Na₂EDTA, 10 mM Tris-HCl, pH 10, 1% of sodium sarcosinate, 1% of triton X-100 and 10% of DMSO). The cell lysis is performed at 4° C. for 60 minutes, followed by denaturation of the DNA at ambient temperature for 20 min in a strongly alkaline solution (1 mM Na₂EDTA and 300 mM NaOH, pH>13.0). After electrophoresis (25 V, 300 mA) for 20 min., the slides are neutralized with the Tris-HCl buffer (0.4 M; pH 7.4) and dehydrated in absolute ethanol or methanol.

4. Microscopic Observation and Image Analysis

The slides are stained with ethidium bromide solution (75 μl of 2 μg/ml) and observed using a BH2-RFL fluorescence microscope (Olympus, Japan) equipped with a 20BG-W dichroic filter (excitation: 515-560 nm; emission: 590 nm) and with an Apo D-Plan 20× objective. The image analysis is carried out with a high sensitivity monochrome CCD camera (Cohu 4912-5000) coupled to a Matrox IP-8 acquisition board. The entire assembly is controlled by means of the Fenestra Komet software (Kinetic Imaging, Liverpool, UK, version 3.1).

A total of 100 cells per sample (50 cells/slide) is analyzed. The parameter used is “tail DNA”, which is defined as the percentage of DNA in the tail of the “comet”. For each series of experiments, a negative control (nonirradiated cells) and a positive control (irradiated cells without composition tested) are included.

5. Statistical Analysis

Nonlinear regressions based on a χ² function are calculated directly from the TM (tail moment) distribution frequencies for each sample. Specifically, Bauer et al. [3] have recently shown that these distributions follow a χ² function. This method is based on an analysis of the distribution according to a χ² law.

The factor n (also called ω² TM), which represents the degree of freedom of the function, is directly correlated with the degree of damage (mean TM). The factor n varies from 2 (intact cells) to 15 (cells extremely damaged, with a Gaussian distribution frequency).

The degree of freedom (n) can be used as an indicator of DNA damage. The distribution frequencies are calculated with the Excel 97 tabulator (Microsoft) and the nonlinear regressions are calculated with the Table Curve 2D software (Jandel Scientific, version 5.0).

### 6. REFERENCES

[1] De Méo M, M. Laget M, Castegnaro M, Duménil G. Genotoxic activity of potassium permanganate acidic sodium. Mutation Res. 1991; 260; 295-306.
[2] Klaude M, Ericksson S, Nygren J, Annstrom G. The comet assay: mechanism and technical consideration. Mutation Res. 1996; 363; 89-96.
[3] Bauer E, Recknagel R D, Fiedler U, Wollweber L, Bock C, Greulich K. O. The distribution of the tail moments in single cell electrophoresis (comet assay) obeys a chi-square (χ²) not a gaussian distribution. Mutation Res. 1998;-398: 101-110.
   7. Degree of Protection by the Compositions Tested

The results according to the table below are obtained.

| | | Degree of |
|---|---|---|
| Composition | OTM-χ² | protection (%) |
| NI | 2.08 ± 0.02 | — |
| NI + ATP (4 mM) | 2.08 ± 0.02 | — |
| NI + Citru (4 mM) | 2.06 ± 0.02 | — |
| NI + ATP (4 mM) + Citru | 2.07 ± 0.02 | — |
| (4 mM) | | |
| UVA | 9.16 ± 0.32 | 0.00% |
| UVA + ATP (4 mM) | 6.31 ± 0.38 | 14.6% |
| | | (NS) |
| UVA + Citru (4 mM) | 2.22 ± 0.20 | 67.7% |
| UVA + ATP (4 mM) + Citru | 2.11 ± 0.04 | 99.6% |
| (4 mM) | | |

OTM-χ² = tail moment χ²: degree of freedom of the function calculated by nonlinear regression of the normalized frequency of distribution of the OTMs. The probability of the models in all cases is P < 0.001.
NI: nonirradiated melanocytes.
NI + ATP: nonirradiated melanocytes pretreated with ATP (4 mM) for 30 min.
NI + Citru: nonirradiated melanocytes pretreated with citrullyl arginine (4 mM) for 30 min.
UVA: melanocytes irradiated with UVA radiation (0.8 J/cm²).
UVA + ATP: melanocytes irradiated with UVA radiation (0.8 J/cm²) and pretreated with ATP (4 mM) for 30 min.
UVA + Citru: melanocytes irradiated with UVA radiation (0.8 J/cm²) and treated with citrullyl arginine (4 mM) for 30 min.
NS: difference not significant between UVA + Citru and UVA + ATP.

The degree of protection by citrullyl arginine is much greater than that by ATP. The synergy between the ATP molecule and the peptide is very clear.

The examples that follow illustrate the invention without, however, limiting the scope thereof.

### EXAMPLES

### Example 1

### Antiwrinkle Cream

| | |
|---|---|
| Sucrose stearate | 0.5-6% |
| Sucrose distearate | 0.5-6% |
| ATP disodium salt | 0.01-0.05% |
| Diguanosine tetraphosphate (Gp4G) | 0.5-1% |
| Caprylic/capric triglyceride | 3-15% |
| Caprylic/capric/succinic triglyceride | 3-15% |
| Ceramides 3 | 0.05-1% |
| Ascorbyl palmitate | 0.01-0.1% |
| Tocopheryl acetate | 0.05-1% |
| Urea | 0.5-2% |
| Calcium chloride | 0.05-0.5% |
| Magnesium chloride | 0.05-0.5% |
| β-Alanyl-L-histidine (Carnosine) | 0.5-1% |
| Gly-His-Lys (peptide powder) | 10-5 ppm |
| Serine | 0.2-2% |
| Glycerol | 0.5-3% |
| Citric acid | 0.1-0.5% |
| Trisodium citrate | 0.5-1.5% |
| Vitamin A palmitate | 0.1-0.5% |
| Phospholipids | 0.1-0.4% |
| Superoxide dismutase | 0.5-2% |
| Sodium hyaluronate | 0.5-3% |
| Potassium sorbate | 0.2-0.5% |
| Sclerotium gum | 0.1-0.5% |
| Xanthan gum | 0.1-0.5% |
| Water | qs 100% |
| Fragrance | qs |
| Preserving agents | qs |

### Example 2

### Moisturizing Milk

| | |
|---|---|
| Diguanosine tetraphosphate (Gp4G) | 0.5-1% |
| Phospholipids | 3% |
| Ceramide 3 | 0.1% |
| Vitamin A palmitate | 0.15% |
| Steareth-20 | 0.2% |
| Steareth-2 | 1 to 3% |
| Methyl paraben | 0.25% |
| Calcium chloride | 0.01% |
| Magnesium chloride | 0.01% |
| Water | qs 100% |
| Cetostearyl alcohol | 2 to 4% |
| Myristyl myristate | 2 to 4% |
| Isopropyl myristate | 4% |
| Glycerol | 1% |
| L-Citrullyl-L-arginine | 0.1 to 2% |
| Dimethicone | 0.5% |
| Lanolinic alcohols | 0.5% |
| Propyl paraben | 0.25% |

### Example 3

### Moisturizing Cream

| | |
|---|---|
| ATP disodium salt | 0.01-0.05% |
| Sorbitan oleate | 3.5% |
| Polysorbate 80 | 2 to 4% |
| Wheatgerm oil | 3% |
| Sweet almond oil | 5% |
| Isopropyl myristate | 12% |
| Phospholipids | 0.5% |
| Ceramide 3 | 0.1% |
| Polyacrylamide & C₁₄₋₁₃ isoparaffin & | 2 to 3.5% |
| Laureth-7 | |
| Vitamin A palmitate | 0.1% |
| Tocopherol | 0.05% |
| Sodium PCA | 0.5% |
| Diguanosine tetraphosphate (Gp4G) | 0.5-1% |
| Sodium hyaluronate | 0.1% |
| Water | qs 100% |
| L-Citrullyl-L-arginine | 0.1 to 2% |
| Preserving agents | qs |
| Fragrance | qs |

### Example 4

### Protective Suntan Cream

| | |
|---|---|
| ATP Disodium salt | 0.01-0.05% |
| Octyl methoxycinnamate | 6.00% |
| (Neo Heliopan AV) | |
| Butylmethoxydibenzoylmethane | 3.00% |
| (Parsol 1789) | |
| Octyltriazone | 2.00% |
| (Uvinul T150) | |
| Di (C₁₂₋₁₃) alkyl tartrate | 8.00% |
| (Cosmacol ETI) | |
| Cetyl alcohol | 0.50% |
| Dimethicone | 0.50% |
| Coco caprylate/caprate | 8.00% |
| PVP/eicosene copolymer | 2.00% |
| Potassium cetyl phosphate | 2.00% |
| Methyl and propyl paraben | 0.25% |
| Disodium EDTA | 0.10% |
| BHT | 0.05% |
| Carbomer | 10.00% |
| Diguanosine tetraphosphate (Gp4G) | 0.5-1% |
| Alanyl-L-histidine (Carnosine) | 0.8-1% |
| Propylene glycol | 5.00% |
| Potassium hydroxide | 4.05% |
| Phenylbenzimidazolesulfonic acid | 2.00% |
| (Eusolex 232) | |
| Tocopheryl acetate | 2.50% |
| Panthenol | 1.00% |
| MSH (Ala-His-Lys-Phe-Tyr) | 0.0001-0.00001% |
| Photolyase | 0.1% |
| Water | qs 100% |
| Fragrance | qs |

### Example 5

### Photoprotective and Repair Suncream

| | |
|---|---|
| ATP Disodium salt | 0.01-0.05% |
| Ethoxydiglycol and cucumber | 8.00% |
| Di-C₁₂₋₁₃ alkyl tartrate | 5.00% |
| (Cosmacol ETI) | |
| Octyl methoxycinnamate | 5.00% |
| (Parsol MCX) | |
| Butylmethoxydibenzoylmethane | 2.00% |
| (Parsol 1789) | |
| Dimethicone trimethylsiloxysilicate | 3.00% |
| Tocopheryl acetate | 0.20% |
| Sucrose distearate | 5.00% |
| Hexylene glycol | 5.00% |
| Butyl, methyl, propyl | 0.40% |
| paraben + phenoxyethanol | |
| L-Citrullyl-L-arginine | 0.1 to 2% |
| Water | qs 100% |
| Diguanosine tetraphosphate (Gp4G) | 1-1.5% |
| MSH (Ala-His-Lys-Phe-Tyr) | 0.0001-0.00001% |
| Endonuclease | 0.2% |
| Alanyl-L-histidine (Carnosine) | 0.5-1% |
| Fragrance | qs |

### Example 6

### Soothing Photorepair Milk

| | |
|---|---|
| Mineral oil | 2.00% |
| Diguanosine tetraphosphate (Gp4G) | 0.5-1% |
| Di-C₁₂₋₁₃ alkyl tartrate | 4.00% |
| (Cosmacol ETI) | |
| Octyl stearate | 3.00% |
| Isoamyl-p-methoxycinnamate | 5.00% |
| (Parsol MCX) | |
| Butylmethoxydibenzoylmethane | 1.00% |
| (Parsol 1789) | |
| Polyglyceryl-3 diisostearate | 4.00% |
| Glyceryl PEG-20 laurate | 1.00% |
| Carbomer | 0.4% |
| Propylene glycol | 2.00% |
| Preserving agents | 0.50% |
| Xanthan gum | 0.30% |
| Triethanolamine | 0.85% |
| Phenylbenzimidazolesulfonic acid | 2.5% |
| (Neo Heliopan Hydro) | |
| Acetyl tyrosine | 2.00% |
| Alanyl-L-histidine (Carnosine) | 0.5-1% |
| Gly-His-Lys (peptide powder) | 10-5 ppm |
| Water | qs 100% |
| Fragrance | qs |

### Example 7

### Desensitizing Face Cream

| | |
|---|---|
| Caprylic/capric/succinic triglyceride | 1 to 10% |
| Ascorbyl palmitate | 0.01 to 0.1% |
| Glyceryl stearate | 1 to 5% |
| Stearic acid | 1 to 5% |
| Tocopherol acetate | 0.1 to 1% |
| Carpylic/capric triglyceride | 1 to 15% |
| ATP disodium salt | 0.01-0.05% |
| Pyridoxine | 0.01 to 0.05% |
| Citric acid | 0.1 to 0.5% |
| Zinc gluconate | 0.1 to 1% |
| Trisodium citrate | 1 to 2.5% |
| L-Citrullyl-L-arginine | 0.1 to 2% |
| Diguanosine tetraphosphate (Gp4G) | 0.5-1% |
| Glycerol | 1 to 4% |
| Vitamin A palmitate | 0.01 to 1% |
| d-Panthenol | 0.1 to 1% |
| Rhamnose | 0.1 to 1% |
| L-Fucose | 0.01 to 1% |
| Lactoferrin/lactoperoxidase | 0.01 to 1% |
| Superoxide dismutase | 0.01 to 1% |
| Polyacrylamide/C₁₃₋₁₄ | 0.2 to 1% |
| isoparaffin/laureth-7 | |
| Water | qs 100% |

### Example 8

### Soothing Body Milk

| | |
|---|---|
| Acrylic acid polymer | 0.1-1.5% |
| Glycyrrhetinic acid | 0.1-1% |
| Triethanolamine | 0.1-2% |
| Butylene glycol | 0.5-4% |
| ATP disodium salt | 0.01-0.05% |
| Ascorbyl palmitate | 0.01 to 0.1% |
| Tocopherol acetate | 0.1 to 1% |
| Pyridoxine | 0.01 to 0.05% |
| Citric acid | 0.1 to 0.5% |
| Zinc gluconate | 0.1 to 1% |
| Trisodium citrate | 1 to 2.5% |
| L-Arginine | 0.1 to 2% |
| Vitamine A palmitate | 0.01 to 1% |
| d-Panthenol | 0.1 to 1% |
| L-Fucose | 0.1 to 1% |
| Lactoferrin/lactoperoxidase | 0.01 to 1% |
| Alanyl-L-histidine (Carnosine) | 0.5-1% |
| R-Gly-Gln-Pro-Arg | 15-20 ppm |
| Superoxide dismutase | 0.01 to 1% |
| Potassium sorbate | 0.1 to 0.6% |
| Preserving agents | qs |
| Water | qs 100% |

### Example 9

### Soothing Cream for Greasy Skin

| | |
|---|---|
| Propylene glycol | 1-8% |
| Sorbitan monolaurate | 0.5-5% |
| Dimethicone copolyol | 0.1-5% |
| Salicylic acid | 0.01-0.5 % |
| Disodium EDTA | 0.05-0.5% |
| Diguanosine tetraphosphate (Gp4G) | 0.5-1% |
| ATP disodium salt | 0.01-0.05% |
| Zinc gluconate | 0.01-1% |
| Ascorbyl palmitate | 0.01 to 0.1% |
| Tocopherol acetate | 0.1 to 1% |
| Pyridoxine | 0.01 to 0.05% |
| Citric acid | 0.1-0.5% |
| Sodium chloride | 0.1-1.5% |
| Trisodium citrate | 1 to 2.5% |
| L-Arginine | 0.1 to 2% |
| Vitamin A palmitate | 0.01 to 1% |
| d-Panthenol | 0.1 to 1% |
| Rhamnose | 0.1 to 1% |
| L-Fucose | 0.01 to 1% |
| Lactoferrin/lactoperoxidase | 0.01 to 1% |
| Gly-His-Lys (peptide powder) | 10-5 ppm |
| Superoxide dismutase | 0.01 to 1% |
| Preserving agents | qs |
| Water | qs 100% |

### Example 10

### Makeup-Removing Lotion

| | |
|---|---|
| Diguanosine tetraphosphate (Gp4G) | 1-2% |
| ATP disodium salt | 0.1-0.5% |
| Trisodium citrate | 1 to 2.5% |
| Glycerol | 0.5-3% |
| Hexylene glycol | 4-5% |
| d-Panthenol | 0.1 to 1% |
| Alanyl-L-histidine (Carnosine) | 0.5-1% |
| Preserving agents (methyl paraben and | qs |
| phenoxyethanol) | |
| Water | qs 100% |

### Sequence Description

| | |
|---|---|
| SEQ ID No. 1 | H-Arg-Pro-Lys-Pro-Gln-Gln-Phe-Phe-Gly-Leu-Met-NH₂: (11 A) |
| SEQ ID No. 2 | H-Tyr-Pro-Ser-Lys-Pro-Asp-Asn-Pro-Gly-Glu-Asp-Ala-Pro-Ala- |
| | Glu-Asp-Met-Ala-Arg-Tyr-Tyr-Ser-Ala-Leu-Arg-His-Tyr-Ile- |
| | Asn-Leu-Ile-Thr-Arg-Gln-Arg-Tyr-NH₂: (36 AA) |
| SEQ ID No. 3 | H-Ala-Gly-Cys-Lys-Asn-Phe-Phe-Trp-Lys-Thr-Phe-Thr-Ser- |
| | Cys-OH: (14 AA) |
| SEQ ID No. 4 | pGlu-Leu-Tyr-Glu-Asn-Lys-Pro-Arg-Arg-Pro-Tyr-Ile-Leu-OH: |
| | (13 AA) |
| SEQ ID No. 5 | H-Ala-Cys-Asp-Thr-Ala-Thr-Cys-Val-Thr-His-Arg-Leu-Ala- |
| | Gly-Leu-Leu-Ser-Arg-Ser-Gly-Gly-Val-Val-Lys-Asn-Asn-Phe- |
| | Val-Pro-Thr-Asn-Val-Gly-Ser-Lys-Ala-Phe-NH₂:(37 AA) |
| SEQ ID No. 6 | H-Ala-Cys-Asn-Thr-Ala-Thr-Cys-Val-Thr-His-Arg-Leu-Ala- |
| | Gly-Leu-Leu-Ser-Arg-Ser-Gly-Gly-Met-Val-Lys-Ser-Asn-Phe- |
| | Val-Pro-Thr-Asn-Val-Gly-Ser-Lys-Ala-Phe-NH₂: (37 AA) |
| SEQ ID No. 7 | H-Val-Pro-Leu-Pro-Ala-Gly-Gly-Gly-Thr-Val-Leu-Thr-Lys- |
| | Met-Tyr-Pro-Arg-Gly-Asn-His-Trp-Ala-Val-Gly-His-Leu-Met- |
| | NH₂: (27 AA) |
| SEQ ID No. 8 | H-Arg-Pro-Pro-Gly-Phe-Ser-Pro-Phe-Arg-OH: (9 AA) |
| SEQ ID No. 9 | H-Tyr-Gly-Gly-Phe-Met-Thr-Ser-Glu-Lys-Ser-Gln-Thr-Pro-Leu- |
| | Val-Thr-Leu-Phe-Lys-Asn-Ala-Ile-Ile-Lys-Asn-Ala-Tyr-Lys |
| | Lys-Gly-Glu-OH: (31 AA) |
| SEQ ID No. 10 | H-Tyr-Gly-Gly-Phe-Leu-OH: (5 AA) |
| SEQ ID No. 11 | H-Tyr-Gly-Gly-Phe-Met-OH: (5 AA) |
| SEQ ID No. 12 | Ac-Ser-Tyr-Ser-Met-Glu-His-Phe-Arg-Trp-Gly-Lys-Pro-Val- |
| | NH₂: (13 AA) |
| SEQ ID No. 13 | H-Ser-Tyr-Ser-Met-Glu-His-Phe-Arg-Trp-Gly-Lys-Pro-Val-Gly- |
| | Lys-Lys-Arg-Arg-Pro-Val-Lys-Val-Tyr-Pro-Asn-Gly-Ala-Glu- |
| | Asp-Glu-Ser-Ala-Glu-Ala-Phe-Pro-Leu-Glu-Phe-OH: (39 AA) |
| SEQ ID No. 14 | H-Ser-Arg-Thr-His-Arg-His-Ser-Met-Glu-Ile-Arg-Thr-Pro-Asp- |
| | Ile-Asn-Pro-Ala-Trp-Tyr-Ala-Ser-Arg-Gly-Ile-Arg-Pro-Val-Gly- |
| | Arg-Phe-NH₂: (31 AA) |

1. A cosmetic composition comprising a bioactive system that combines, firstly, a stable form, in aqueous solution, of ATP (adenosine triphosphate) with, optionally, an ATP precursor, for example Gp₄G (diguanosine tetraphosphate) or Ap₄A (diadenosine tetraphosphate) and, secondly, at least one biomimetic peptide comprising at most six amino acids, that mimics a cutaneous polypeptide or a cutaneous protein, or a biomolecule that is an agonist or antagonist with respect to said polypeptide or to said protein.

2. The composition as claimed in claim 1, characterized in that the stable form is a sodium salt of ATP, for example a disodium salt.

3. The composition as claimed in claim 1, characterized in that the biomimetic peptide is functionally active in the biosynthesis of a structural molecule of the skin, or of an enzyme present in the skin.

4. The composition as claimed in claim 1, characterized in that the biomimetic peptide is functionally active in the transfer of information in the skin, and is, for example, a biologically active fraction of a hormone or cytokine present in the skin.

5. The composition as claimed in claim 1, characterized in that the biomimetic peptide is chosen from the group consisting of histidine-β-alanyl, the peptide R-Gly-Gln-Pro-Arg, the peptide Tyr-Arg, the peptide R-Lys-Thr-Thr-Lys-Ser, N-acetyl-Tyr-Arg-R, the peptide Lys-Thr-Thr-Lys-Ser, the peptide Ala-Arg-His-Leu-Phe-Tyr (or alpha-MSH), and the peptide Gly-Gln-Asp-Pro-Val-Lys (or elafin); R being any amino acid.

6. The composition as claimed in claim 1, characterized in that the biomimetic peptide is a dipeptide.

7. The composition as claimed in claim 6, characterized in that the dipeptide corresponds to the formula Arg-R or His-R, in which R is any amino acid.

8. The composition as claimed in claim 6, characterized in that the dipeptide is in the form of an oligomer, of formula (R—R)ₙ, with 1<n<3.

9. The composition as claimed in claim 8, characterized in that the dipeptide corresponds to the formula (Arg-Lys)ₙ, with 1<n<3.

10. The composition as claimed in claim 1, characterized in that the biomimetic peptide is a tripeptide.

11. The composition as claimed in claim 10, characterized in that the tripeptide is Gly-His-Lys or Gly-Glu-Pro.

12. The composition as claimed in claim 1, characterized in that the biomimetic peptide is a tetrapeptide.

13. The composition as claimed in claim 12, characterized in that the tetrapeptide is Leu-Pro-Thr-Val or Lys-Thr-Ser-R or Gly-Glu-Pro-R; R being any amino acid.

14. The composition as claimed in claim 1, characterized in that the biomimetic peptide is a pentapeptide, for example Val-Ala-Lys-Leu-R; R being any amino acid.

15. The composition as claimed in claim 1, characterized in that the biomimetic peptide is a hexapeptide.

16. The composition as claimed in claim 15, characterized in that the biomimetic peptide is Ala-R₁-R₂-R₃-Phe-Try, with R₁, R₂ and R₃ each equal to any amino acid.

17. The composition as claimed in claim 1, characterized in that it comprises an amino acid, for example chosen from the group consisting of creatine, decarboxy carnosine and a glutamine, for example N-acetylglutamine.

18. The composition as claimed in claim 1, characterized in that it comprises a protein, for example chosen from the group consisting of superoxide dismutase, endonucleases, photolyase and cytokines from milk.

19. The composition as claimed in claim 1, characterized in that the bioactive system represents at most 10%, and preferably between 1% and 10⁻⁷%, by weight of said composition.

20. The composition as claimed in claim 1, characterized in that it comprises at least one cosmetic active principle potentiated by the bioactive system.

21. The composition as claimed in claim 1, characterized in that it is in the form of a water-in- oil or oil-in-water emulsion, the bioactive system being included in the aqueous phase.

22. The composition as claimed in claim 1, characterized in that the bioactive system, the ATP and, optionally, the ATP precursor represent at most 10%, and preferably between 0.01% and 5%, by weight.
